(19) United States
(12) Patent Application Publication
(10) Pub. No.: US 2007/0099996 A1
(43) Pub. Date: May 3, 2007

(54) Pharmaceutical compositions of acitretin

(76) Inventor: Shashikanth Isloor, Shimoga District, Karnataka (IN)

Correspondence Address: Jayadeep R. Deshmukh, Ranbaxy Inc., 600 College Road East, Suite 2100, Princeton, NJ, 08540, US

(21) Appl. No.: 10/557,572

(22) PCT Filed: May 21, 2004
(86) PCT No.: PCT/IB04/01636

§ 371 Date: Nov. 27, 2006

(30) Foreign Application Priority Data
May 20, 2003 (IN) 713/DEL/2003

(51) Int. Cl. A61K 31/192

(52) U.S. Cl. 514/570

(57) ABSTRACT

The present invention relates to stable pharmaceutical compositions of acitretin with significantly enhanced bioavailability characteristics. The pharmaceutical compositions include a micronized solid dispersion of acitretin with one or more surface modifiers. Also provided are processes of preparing the compositions by aqueous grinding or milling of acitretin in presence of a hydrophilic surface modifier.

### TECHNICAL FIELD OF THE INVENTION

The present invention relates to stable pharmaceutical compositions of acitretin with significantly enhanced bioavailability characteristics. Also provided are processes of preparing the compositions by aqueous grinding or milling of acitretin in the presence of a hydrophilic surface modifier.

### BACKGROUND OF THE INVENTION

There is an ever present need in the pharmaceutical industry for improved pharmaceutical formulations that enhance the efficacy of poorly soluble therapeutic agents. The solubility of active ingredients remains one of the most challenging aspects in formulation development. For an oral route of administration it is well known in the art that unless the substance has an aqueous solubility above 10 mg/ml over the pH range 1-7, potential absorption problems may occur. Numerous active ingredients suffer from the disadvantage of being poorly soluble in an aqueous medium and thus have insufficient dissolution profiles and poor bioavailability following oral administration.

Acitretin, a retinoid, is a metabolite of etretinate and is related to both retinoic acid and retinol (Vitamin A). It is commercially available in 10 mg and 25 mg capsules for oral administration and is indicated for the treatment of severe psoriasis including erythromycodermic and generalized pustular types in adults.

Acitretin is practically insoluble in water and has poor wettability in aqueous media. It is sensitive to air, heat and light, especially in solution. It must be stored in an air-tight container, protected from light, at a temperature between 2° and 8° C. Due to its instability and relative insolubility, the bioavailability of the drug after oral administration is low and presents a challenge to a development pharmacist. Therefore, it would be desirable to provide a dosage form in which the drug is stable and predictably bioavailable.

U.S. Pat. No. 4,105,681 discloses acitretin compositions, processes of preparing such compositions, and methods of using acitretin as an anti-tumour agent. The patent further discloses that acitretin can also be useful for topical and systemic therapy of acne, psoriasis and other related dermatological disorders.

U.S. Pat. No. 5,472,954 describes the use of certain polymers in the preparation of cyclodextrin drug complexes as a means for increasing the solubilizing and stabilizing effects of cyclodextrin derivatives on lipophilic drugs, such as acitretin, and complexation therewith.

Several techniques have been used to improve the bioavailability of poorly soluble therapeutically active compounds, such as particle size reduction, solid dispersion, self-emulsifying drug delivery systems and the like.

Based on these techniques, U.S. Pat. No. 4,540,602 discloses a solid drug pulverized in an aqueous solution of a water-soluble high molecular weight substance using wet grinding. As a result, the drug is formed into finely divided particles ranging from 0.5 μm to less than 5 μm in diameter.

It is known that increasing the surface area, i.e. decreasing particle size, can increase the rate of dissolution of drug particles. Particle size reduction can be carried out by two types of milling procedures—dry milling and wet milling. The choice of dry or wet milling depends on the characteristics of the product. If it undergoes physical or chemical changes in an aqueous medium then dry milling is recommended. Conventional dry milling techniques can process drugs into powders that have particle sizes ranging from 0.1 to 50 microns. Wet grinding is beneficial in further reducing particle size, but flocculation restricts the lower limit to approximately 10 microns.

Dosage forms containing micronized drug particles have enhanced dissolution rates and consequently higher bioavailability, but this also creates additional processing problems. Highly micronized drug particles possess poor flow properties and high chances of agglomeration during processing. Moreover, for practically insoluble drugs like acitretin, particle size reduction of the drug will not necessarily result in better bioavailability as there will be less dispersibility due to its poor wetting characteristics.

In order to eliminate these problems, the poorly soluble drug is finely dispersed through-out a dispersion medium which includes a surface modifier. This dispersion is then subjected to size reduction.

The inventors have discovered that stable, dispersible micronized particles of acitretin can be prepared by wet milling a solid dispersion of acitretin and a hydrophilic surface modifier. The micronized particles obtained through this process exhibit improved bioavailability characteristics.

### SUMMARY OF THE INVENTION

In one general aspect there is provided a pharmaceutical composition which includes a micronized solid dispersion of acitretin with one or more surface modifiers.

Embodiments of the composition may include one or more of the following features. For example, the acitretin may have a particle size of d90 less than about 5.0 microns or a particle size of d90 less than about 2.5 microns

The surface modifier may include one or more polymers and natural products. The polymers may be one or more of polyvinyl pyrrolidone, hydroxypropyl methyl cellulose, polyethylene glycols, carboxymethyl cellulose sodium and polyvinyl alcohol. The natural products may be one or more of dextran, xanthan, chitosan, pectin, dextrin, maltodextrin, starch, alginates and pullulan.

In one embodiment the acitretin and the one or more surface modifiers are present at a ratio range of about 1:2 to about 1:10.

The composition may include one or more antioxidants. The antioxidant may be one or more of sodium ascorbate and other salts of ascorbic acid, propyl gallate, alpha tocopherol, butylated hydroxyl toluene, butylated hydroxyl anisole and lecithin.

The composition may also include one or more surfactants. The surfactants may be one or more of sodium lauryl sulphate, sodium laurate, dialkyl sodium sulfosuccinates, potassium stearate and sodium stearate.

The composition may be one or more of tablets, capsules, sachet, granules and dispersible powder.

In another general aspect there is provided a process for preparing a pharmaceutical composition of acitretin. The process includes dispersing acitretin throughout a dispersion medium which includes one or more surface modifiers to form a dispersion or suspension and then wet milling the dispersion or suspension and drying the milled dispersion or suspension.

Embodiments of the process may include one or more of the following features. For example, the acitretin may have a particle size of d90 less than about 5.0 microns or a particle size of d90 less than about 2.5 microns.

The surface modifier may include one or more of polymers and natural products. The polymers may be one or more of polyvinyl pyrrolidone, hydroxypropyl methyl cellulose, polyethylene glycols, carboxymethyl cellulose sodium and polyvinyl alcohol. The natural products may be one or more of dextran, xanthan, chitosan, pectin, dextrin, maltodextrin, starch, alginates and pullulan.

The ratio of acitretin to surface modifier ranges from about 1:2 to about 1:10.

The wet milling may be carried out using one or more of ball mill, attrition mill, vibratory mill, media mill or high pressure homogenizer. The wet milling may be carried out using a media mill which may be a Dyno-mill. The wet milled acitretin particles may be dried in a fluidized bed dryer or spray dryer.

The process may further include blending the wet milled acitretin particles with at least one antioxidant before drying. The antioxidant may include one or more of sodium ascorbate and other salts of ascorbic acid, propyl gallate, alpha tocopherol, butylated hydroxyl toluene, butylated hydroxyl anisole and lecithin.

The process may also include blending the dried particles with one or more surfactants. The surfactants may include one or more of sodium lauryl sulphate, sodium laurate, dialkyl sodium sulfosuccinates, potassium stearate and sodium stearate.

The dried particles may be formulated into one or more of tablets, capsules, sachet, granules and dispersible powder.

In another general aspect there is provided a method of treating psoriasis by administering to a patient in need thereof a pharmaceutical composition which includes a micronized solid dispersion of acitretin with one or more surface modifiers.

Embodiments of the method may include one or more of the features described above or the following. For example, the pharmaceutical composition may further include one or more of methotrexate, calcipotriol and cyclosporin.

The details of one or more embodiments of the inventions are set forth in the description below. Other features, objects and advantages of the inventions will be apparent from the description and claims.

### DETAILED DESCRIPTION OF THE INVENTION

The inventors have developed a pharmaceutical composition of acitretin with improved bioavailability characteristics which includes a micronized solid dispersion of acitretin and one or more surface modifiers. Also developed is a process for preparing a pharmaceutical composition of acitretin with improved bioavailability characteristics which includes wet milling acitretin in the presence of one or more surface modifiers.

Acitretin used in the pharmaceutical compositions described herein can be prepared by any known method, such as, for example, using the procedures disclosed in U.S. Pat. No. 4,105,681 incorporated herein in its entirety by reference.

The mechanical means applied to reduce the particle size of the drug substance can be carried out conveniently in a dispersion mill. Suitable dispersion mills include ball mills, attrition mills, vibratory mills and media mills, such as, bead mills or high pressure homogenisers. A media mill is preferred due to the relatively short milling time required to provide the intended result. Dyno-mill, a continuously operating bead mill with a horizontal grinding container can also be used for dispersion and finest wet grinding. In Dyno-milling, the product to be ground is pumped into the grinding chamber, where it is exposed for a certain period of time to the stress of the moving grinding beads. Before leaving the grinding chamber, the grinding beads are separated from the product by means of a separation system.

The grinding media or beads for particle size reduction can be selected from rigid media preferably spherical or particulate in form having an average size that is less than about 1 mm. Beads may be formed from one or more of zirconium oxide, magnesia, zirconium silicate and glass.

The reduction of the particle size of acitretin to a D₉₀ of less than 5 microns, particularly less than 2.5 microns, results in improved bioavailability of acitretin pharmaceutical compositions as compared to acitretin pharmaceutical compositions that contain larger sized acitretin particles. Acitretin particles having a D₉₀ particle size of less than about 5.0 microns, particularly less than about 2.5 microns are hereinafter referred to as “micronized acitretin particles.” Also used herein, “D₉₀ particle size” is the particle size of at least 90% of the particles of acitretin used in the composition.

Suitable surface modifiers can be selected from organic and inorganic pharmaceutical excipients, such excipients include various polymers and natural products. Particularly, hydrophilic excipients may be selected.

Suitable polymers surface modifiers include one or more of polyvinyl pyrrolidone, hydroxypropyl methyl cellulose, polyethylene glycols, carboxymethyl cellulose sodium and polyvinyl alcohol.

Suitable natural product surface modifiers may include one or more of dextran, xanthan, chitosan, pectin, dextrin, maltodextrin, starch, alginates, pullulan and the like.

The acitretin and one or more surface modifiers are present in a ratio ranging from about 1:2 to about 1:10.

The milled suspension of acitretin and surface modifier in water is dried in a fluidized bed dryer or using spray drier, using a suitable gas, for example air or nitrogen, as the carrier. Additionally, an antioxidant compatible with the active may be incorporated into the milled suspension prior to the spray drying step. Particularly, a water soluble antioxidant may be added. Suitable water-soluble antioxidants include one or more of sodium ascorbate and other salts of ascorbic acid, propyl gallate, alphatocopherol, butylated hydroxyl toluene, butylated hydroxyl anisole, lecithin and the like.

Suitable surfactants or surface active agents may include one or more of sodium lauryl sulphate, sodium laurate, dialkyl sodium sulfosuccinates, potassium stearate, sodium stearate and the like.

The micronized or milled blend is finally formulated into oral dosage forms, such as, tablets, capsules, sachet, granules, dispersible powder, etc. Such oral dosage forms may further include pharmaceutically acceptable excipients. Suitable pharmaceutically acceptable excipients include one or more of diluents, lubricants and glidants that are compatible with acitretin and other excipients.

Suitable diluents may include one or more of lactose, microcrystalline cellulose, mannitol, starch, dextrose, calcium phosphate dihydrate, sucrose and the like.

Suitable lubricants may include one or more of talc, magnesium stearate, calcium stearate, polyethylene glycol, stearic acid, and sodium stearyl fumarate.

Suitable glidants may include one or more of colloidal silicon dioxide, magnesium silicate and talc.

The micronized particles of acitretin were finally filled into hard gelatin capsules of suitable size and these capsules were subjected to accelerated stability studies at 40°±2° C. and 75±5% relative humidity. These were evaluated on the basis of assay, in vitro dissolution, moisture content and related substances measured between initial and 3-month time points.

The following examples further exemplify the invention and are not intended to limit the scope of the invention.

### EXAMPLE 1

In order to evaluate the most suitable method for improving bioavailability of acitretin, many different approaches were tried. These were air-jet milling, solid-solid dispersion, granulation with a surfactant and wet-milling. Capsules containing acitretin were prepared using all the above-mentioned methods as follows:

A. Capsules including air-jet milled acitretin

B. Capsules including a solid-solid dispersion of acitretin with maltodextrin

C. Capsules including acitretin granulated with surfactant

D. Capsules including acitretin wet-milled with maltodextrin

E. Soft gelatin capsules including oil based wet-milled acitretin

Capsules prepared according to A, B, C, D and E processes were subjected to the following studies:

1. Disintegration time

2. Assay

3. Uniformity of content

4. Dissolution profile

These capsules also were compared with the marketed formulation (Soriatane capsules, Roche).

TABLE 1
Disintegration time, assay and uniformity of content of acitretin capsules prepared
according to A, B, C, D and E as compared to the innovator's capsules (R) (Soriatane
capsules, 25 mg, Roche).

| | | | Granulation | | | Innovator |
|---|---|---|---|---|---|---|
| | Air-jet | Solid | with | | Oil- | marketed |
| | milled | dispersion | surfactant | Wet-milling | milling | formulation |
| Test | A | B | C | D | E | R |
| Disintegration | 6 | 5-6 | 5-6 | 5-6 | — | 5-6 |
| time (minutes) | | | | | | |
| Assay (mg) | 24.18 | 24.56 | 25.31 | 25.30 | 24.932 | 25.33 |
| Uniformity of | 95.7 | 101.6 | 99.8 | 100.03 | 99.73 | 101.32 |
| content (%) | | | | | | |

TABLE 2
Dissolution profile of Acitretin capsules prepared according
to A, B, C and D as compared to the innovator's capsules
(R) (Soriatane capsules, 25 mg, Roche) in 900 ml of 3% w/v
sodium lauryl sulphate, pH 9.6, USP Apparatus 1 at 100 rpm, 37° C.

| | Percent acitretin | | | | |
|---|---|---|---|---|---|
| Time | released (%) | | | | |
| (Minutes) | A | B | C | D | R |
| 10 | 98 | 86 | 87 | 32 | 92 |
| 20 | 99 | 95 | 92 | 56 | 98 |
| 30 | 102 | 99 | 99 | 99 | 102 |

All the formulations were subjected to pharmacokinetic studies in human volunteers to ascertain the in vivo performance of each of the formulation in comparison to the innovator's formulation. The results of the study are reported in Table 3.

TABLE 3
Pharmacokinetic parameters obtained with capsules prepared according to A, B, C, D and
E with the innovator's capsules (R)

| | Parameters | | | | | |
|---|---|---|---|---|---|---|
| | A | B | C | D | E | R |
| | Number of subjects | | | | | |
| | 11 | 6 | 4 | 12 | 6 | 12 |
| T(max) hours | 3.237 | 3.118 | 4.025 | 2.74 | 3.133 | 2.486 |
| (Geometric | (1.5-6.0) | (2.5-4.0) | (3.0-5.0) | (1.5-4.5) | (2.0-5.0) | (1.5-4.5) |
| mean) | | | | | | |
| Cmax (ng/ml) | 99.967 | 36.816 | 38.893 | 253.957 | 63.577 | 206.389 |
| (Geometric | (86.29-171.63) | (13.42-75.60) | (12.67-72.37) | (97.85-733.75) | (24.51-177.75) | (49.24-437.77) |
| mean) | | | | | | |
| AUC_{(O-t)} | 434.934 | 116.178 | 101.536 | 1487.107 | 248.416 | 1073-397 |
| (ng · hr/ml) | (111.96-969.48) | (14.66-314.87) | (9.16-305.17) | (500.95-5665.87) | (78.24-767.10) | (392.96-2292.14) |
| (Geometric | | | | | | |
| mean) | | | | | | |
| AUC_{(O-α)} | 526.235 | 166.304 | 262.048 | 1734.481 | 330.653 | 1185.101 |
| (ng · hr/ml) | (210.55-1063.72) | (55.84-359.58) | (216.51-338.93) | (468.10-6370.94) | (105.06-882.77) | (538.98-2292.14) |
| (Geometric | | | | | | |
| mean) | | | | | | |

From the results, it was concluded that wet-milled acitretin (D) exhibited better absorption when compared with acitretin in marketed formulation (R), air-jet milled acitretin (A), in solid dispersion with maltodextrin (B), acitretin granulated with surfactant (C) and oil-milled acitretin (E).

Comparative particle size distribution of unmilled acitretin, air-jet milled acitretin, oil-milled and wet milled solid dispersion of acitretin.

TABLE 4

| Particle | | Air-jet | | Wet-milled solid |
|---|---|---|---|---|
| size | Unmilled | milled | Oil-milled | dispersion of |
| (microns) | acitretin | acitretin | acitretin | acitretin |
| D₁₀ | 11.14 | 2.13 | 0.110 | 0.073 |
| D₅₀ | 39.59 | 5.51 | 3.140 | 0.156 |
| D₉₀ | 105.0 | 14.48 | 13.796 | 2.401 |

### EXAMPLE 2

A solid-solid dispersion of acitretin was fabricated using maltodextrin and the dispersion was wet milled with purified water in a dyno-mill:

| S. No. | Ingredients | Quantity/lot in gm |
|---|---|---|
| 1 | Acitretin | 100 |
| 2 | Maltodextrin | 300 |
| 3 | Purified water | 800 |

Process:
1. Maltodextrin was dissolved in purified water.
2. Acitretin was weighed and added to the solution of Step 1 and dispersed.
3. The dispersion was Dyno milled.

4. The following components were dissolved and added to the dispersion of Step 3.

| S. No. | Ingredients | Quantity/lot in gm |
|---|---|---|
| 1 | Sodium ascorbate | 50 |
| 2 | Purified water | 200 |

5. The dispersion of step 4 was spray dried using a Buchi Lab Model Spray Drier, using nitrogen as the carrier gas.

6. Spray-dried acitretin was blended with the following ingredients and filled into capsules:

| S. No. | Ingredients | Quantity/lot in gm |
|---|---|---|
| 1 | Acitretin spray dried* (Step 6) | 56.25 |
| 2 | Colloidal silicon dioxide | 6.0 |
| 3 | Sodium lauryl sulphate | 12.5 |
| 4 | Microcrystalline cellulose | 25.25 |

*equivalent to 12.5 gm acitretin

The blend was filled in size 1 hard gelatin capsules using a manual capsule filing machine.

Milling Conditions:

| | |
|---|---|
| Beads size range | 0.4-0.6 mm |
| Beads Type | Zirconium |
| Beads quantity | 480 ml (80% of mill chamber) |
| Agitator shaft speed | 2000 rpm |
| Milling mode | Continuous recirculation |
| Peristaltic pump pressure | 0.2-0.4 bar |
| Flow rate of dispersion | 12-14 gms/sec. |
| Mill pressure | 1.5 bar |
| Cooling media | Water continuous circulation |
| Product temperature | 38-42° C. |
| Milling time | 60 minutes |

### EXAMPLE 3

### Preparation of Capsules Containing 10 Mg of Acitretin

| S. No. | Ingredients | Quantity/lot in gm |
|---|---|---|
| 1 | Acitretin* | 40 |
| 2 | Maltodextrin | 120 |
| 3 | Purified water | 320 |

*equivalent to 5 gm acitretin

Process: The process followed is similar to that followed in Example 2.

| S. No. | Ingredients | Quantity/lot in gm |
|---|---|---|
| 1 | Sodium ascorbate | 20 |
| 2 | Purified water | 80 |

| S. No. | Ingredients | Quantity/lot in gm |
|---|---|---|
| 1. | Acitretin (spray dried) | 22.5 |
| 2. | Colloidal silicon dioxide | 2.0 |
| 3. | Sodium lauryl sulphate | 5.0 |
| 4. | Microcrystalline cellulose | 10.1 |

The blend obtained was filled into size 4 hard gelatin capsules using a manual capsule filling machine.

### EXAMPLE 4

| S. No. | Ingredients | Quantity in mg |
|---|---|---|
| 1 | Acitretin | 25 |
| 2 | Maltodextrin | 75 |
| 3 | Purified water | qs |

Process:
1. Maltodextrin was dissolved in purified water.
2. Acitretin was weighed and added to the solution of Step 1 and dispersed.
3. The dispersion was Dyno milled.
4. The dispersion of step 4 was spray dried.

5. Spray-dried acitretin was blended with the following ingredients and filled into capsules:

| S. No. | Ingredients | mg/capsule |
|---|---|---|
| 1 | Acitretin spray dried* (Step 4) | 100.0 |
| 2 | Colloidal silicon dioxide | 12.0 |
| 3 | Sodium lauryl sulphate | 25.0 |
| 4 | Microcrystalline cellulose | 63.0 |

*equivalent to 25 mg acitretin

The blend was filled in size 1 hard gelatin capsules using a manual capsule filing machine.

### EXAMPLE 5

Accelerated Stability Studies

The chemical and physical stability of packaged acitretin capsules under accelerated stability conditions of 40°±2° C. and 75±5% relative humidity, were evaluated on the basis of assay, in vitro dissolution, moisture content and related substances measured between initial and 3-month time points. The capsules were prepared according to Example 4.

Container: 60 CC HDPE bottle with CR closure, Pack: 30's.

Container: 150 CC HDPE bottle with CR closure, Pack: 120's.

TABLE 5

| | Parameters | |
|---|---|---|
| | | Moisture content |
| Time | Assay (mg/capsule) | (% w/w) |

| (Months) | 30's pack | 120's pack | 30's pack | 120's pack |
|---|---|---|---|---|
| 0 | 25.5 | 25.5 | 5.1 | 5.1 |
| 1 | 25.1 | 24.9 | 5.3 | 5.1 |
| 2 | 24.7 | 24.7 | 4.3 | 4.4 |
| 3 | 24.9 | 24.8 | 4.6 | 4.7 |

TABLE 6
In vitro dissolution (900 ml 3% w/w sodium lauryl sulphate,
pH 9.6, USP Apparatus 1, 100 rpm, 37° C.)

| | Percent Acitretin released (%) | | | | | | | |
|---|---|---|---|---|---|---|---|---|
| | 10 | | 20 | | 30 | | 45 | |
| Time | Minutes | | Minutes | | Minutes | | Minutes | |
| (Months) | 30's | 120's | 30's | 120's | 30's | 120's | 30's | 120's |
| 0 | 23.5 | 23.5 | 61.7 | 61.7 | 85.0 | 85.0 | 98.3 | 98.3 |
| 1 | 24.0 | 23.7 | 61.8 | 62.5 | 83.7 | 81.8 | 99.7 | 98.5 |
| 2 | 25.2 | 22.8 | 62.0 | 62.7 | 82.8 | 84.2 | 96.7 | 96.8 |
| 3 | 19.3 | 21.2 | 63.2 | 61.2 | 83.9 | 86.2 | 96.4 | 97.6 |

TABLE 7
Related substances (% w/w)

| | Related substances (% w/w) | | | | | | | |
|---|---|---|---|---|---|---|---|---|
| | | | Any other | | Any | | | |
| | Impurity | | known | | Unknown | | Total | |
| Period | A | | impurity | | impurity | | impurity | |
| (Months) | 30's | 120's | 30's | 120's | 30's | 120's | 30's | 120's |
| 0 | 0.0666 | 0.066 | ND | ND | 0.067 | 0.067 | 0.133 | 0.133 |
| 1 | 0.068 | 0.080 | ND | ND | 0.073 | 0.080 | 0.141 | 0.160 |
| 2 | 0.063 | 0.078 | ND | ND | 0.093 | 0.087 | 0.156 | 0.165 |
| 3 | 0.063 | 0.060 | ND | ND | 0.090 | 0.095 | 0.153 | 0.155 |

ND = Not detected

The above examples illustrate various embodiments and they are not to be construed to limit the claims in any manner. Moreover, while several particular forms of the inventions have been described, it will be apparent that various modifications and combinations of the inventions detailed in the text can be made without departing from the spirit and scope of the inventions. Accordingly, it is not intended that the inventions be limited, except as by the appended claims.

We claim:

1. A pharmaceutical composition comprising a micronized solid dispersion of acitretin with one or more surface modifiers.

2. The composition according to claim 1, wherein the acitretin has a particle size of d₉₀ less than about 5.0 microns.

3. The composition according to claim 1, wherein the acitretin has a particle size of d₉₀ less than about 2.5 microns.

4. The composition according to claim 1, wherein the surface modifier comprises one or more polymers and natural products.

5. The composition according to claim 4, wherein the one or more polymers comprise one or more of polyvinyl pyrrolidone, hydroxypropyl methyl cellulose, polyethylene glycols, carboxymethyl cellulose sodium and polyvinyl alcohol.

6. The composition according to claim 4, wherein the natural products comprise one or more of dextran, xanthan, chitosan, pectin, dextrin, maltodextrin, starch, alginates and pullulan.

7. The composition according to claim 1, wherein the acitretin and the one or more surface modifiers are present at a ratio range of about 1:2 to about 1:10.

8. The composition according to claim 1, further comprising one or more antioxidants.

9. The composition according to claim 8, wherein the one or more antioxidant comprise one or more of sodium ascorbate and other salts of ascorbic acid, propyl gallate, alpha tocopherol, butylated hydroxyl toluene, butylated hydroxyl anisole and lecithin.

10. The composition according to claim 1, further comprising one or more surfactants.

11. The composition according to claim 10, wherein the one or more surfactants comprise one or more of sodium lauryl sulphate, sodium laurate, dialkyl sodium sulfosuccinates, potassium stearate and sodium stearate.

12. The composition according to claim 1, wherein the composition comprises one or more of tablets, capsules, sachet, granules and dispersible powder.

13. A process for preparing a pharmaceutical composition of acitretin, the process comprising the steps of:
a. dispersing acitretin throughout a dispersion medium comprising one or more surface modifiers to form a dispersion or suspension;
b. wet milling the dispersion or suspension; and
c. drying the milled dispersion or suspension.

14. The process according to claim 13, wherein the acitretin has a particle size of d₉₀ less than about 5.0 microns.

15. The process according to claim 13, wherein the acitretin has a particle size of d₉₀ less than about 2.5 microns.

16. The process according to claim 13, wherein the surface modifier comprises one or more of polymers and natural products.

17. The process according to claim 16, wherein the one or more polymers comprise one or more of polyvinyl pyrrolidone, hydroxypropyl methyl cellulose, polyethylene glycols, carboxymethyl cellulose sodium and polyvinyl alcohol.

18. The process according to claim 16, wherein the one or more natural products comprise one or more of dextran, xanthan, chitosan, pectin, dextrin, maltodextrin, starch, alginates and pullulan.

19. The process according to claim 13, wherein the ratio of acitretin to surface modifier ranges from about 1:2 to about 1:10.

20. The process according to claim 13, wherein the wet milling is carried out using one or more of ball mill, attrition mill, vibratory mill, media mill or high pressure homogenizer.

21. The process according to claim 20, wherein the wet milling is carried out using a media mill comprising a Dyno-mill.

22. The process according to claim 13, wherein the wet milled acitretin particles are dried in a fluidized bed dryer or spray dryer.

23. The process according to claim 13, further comprising blending the wet milled acitretin particles with at least one antioxidant before drying.

24. The process according to claim 23, wherein the antioxidant comprises one or more of sodium ascorbate and other salts of ascorbic acid, propyl gallate, alpha tocopherol, butylated hydroxyl toluene, butylated hydroxyl anisole and lecithin.

25. The process according to claim 13, further comprising blending the dried particles with one or more surfactants.

26. The process according to claim 25, wherein the one or more surfactants comprise one or more of sodium lauryl sulphate, sodium laurate, dialkyl sodium sulfosuccinates, potassium stearate and sodium stearate.

27. The process according to claim 13, further comprising formulating the dried particles into one or more of tablets, capsules, sachet, granules and dispersible powder.

28. A method of treating psoriasis by administering to a patient in need thereof a pharmaceutical composition comprising a micronized solid dispersion of acitretin with surface modifier.

29. The method according to claim 28, wherein the pharmaceutical composition further comprises one or more of methotrexate, calcipotriol and cyclosporin.
